(19) United States
(12) Patent Application Publication
(10) Pub. No.: US 2023/0154304 A1
(43) Pub. Date: May 18, 2023

(54) SYSTEMS AND METHODS FOR ACTIVITY TRACKING AND PHYSIOLOGICAL SENSING FOR CARDIAC RECOVERY ASSESSMENT AND SUPPORT IN MECHANICAL BLOOD PUMPS

(71) Applicant: ABIOMED, Inc., Danvers, MA (US)

(72) Inventor: Alexander Ship, Danvers, MA (US)

(73) Assignee: ABIOMED, Inc., Danvers, MA (US)

(21) Appl. No.: 17/988,840

(22) Filed: Nov. 17, 2022

Related U.S. Application Data
(60) Provisional application No. 63/280,327, filed on Nov. 17, 2021.

(51) Int. Cl. G08B 21/04; G16H 40/67

(52) U.S. Cl. CPC G08B 21/0453; G16H 40/67; A61B 2562/0219

(57) ABSTRACT

Disclosed is a system and method for supporting and monitoring mechanical blood pumps in use with a subject. The system includes (i) a mechanical blood pump; (ii) sensors configured to detect a movement and a physiological condition of the subject; and (iii) one or more processors. The one or more processors may be configured to enable the adjustment of the support provided by the blood pump, such as via the following steps: (i) receiving input from the one or more sensors; (ii) determining an activity type, an activity intensity, or both an activity type and intensity based on the input; (iii) determining a value representative of cardiac recovery based on the input and the determined activity type, intensity, or both type and intensity; and (iv) generating a response based on the determinations and/or the received input.

### CROSS-REFERENCE TO RELATED APPLICATIONS

The present application claims priority to U.S. Provisional Application No. 63/280,327, filed Nov. 17, 2021, the entirety of which is incorporated by reference herein.

### TECHNICAL FIELD

The present disclosure relates to support systems for subjects recovering from cardiac treatments, and specifically to systems and methods for incorporating data from signals from a blood pump, as well as signals from other components, to aid in tracking recovery of a subject

### BACKGROUND

To enable a previously damaged human heart to recover, the pulsation of the heart may be supported by means of a mechanical circulatory support device, such as an artificial pump. Intravascular blood pumps may provide hemodynamic support and facilitate heart recovery. Intravascular blood pumps may be inserted into, e.g., the heart and supplement cardiac output in parallel with the native heart to provide supplemental cardiac support to subjects with cardiovascular disease. An example of such a device is the IMPELLA® family of devices (Abiomed, Inc., Danvers Mass.).

Currently, it is difficult for clinicians to determine the amount of support a device should deliver or when to terminate use of a cardiac assist device. Thus, clinicians tend to rely on qualitative judgments and indirect estimates of cardiac function, such as measuring intracardiac or intravascular pressures using fluid filled catheters. Such qualitative judgments are intermittent, indirect, and inconsistent, and are not capable of being done in real-time.

### BRIEF SUMMARY

Various deficiencies in the prior art are addressed below by the disclosed systems and techniques. For example, the disclosed systems and methods may provide a continuous assessment of recovery and provide clinical insights to the supervising physicians on an ongoing and remote basis. Such techniques may also provide an ongoing qualitative measure of quality of life and partial heart failure classification, e.g., is the subject becoming more active as a result of the mechanical circulator support (MCS) device.

Disclosed is a system for support modulation in mechanical blood pumps for use with a subject. The system may include (i) a mechanical blood pump; (ii) one or more sensors configured to detect a movement and a physiological condition of the subject; and (iii) one or more processors. The one or more processors may be configured to, e.g., generate a response based on the received data, such as enabling the adjustment of the support provided by the blood pump or directing medical personnel to a potential issue. This may be done via several steps, including: (i) receive input from the one or more sensors; (ii) determine an activity type, an activity intensity, or both an activity type and intensity based on the input; (iii) determine a first value representative of cardiac recovery based on the input and the determined activity type, intensity, or both type and intensity; and (iv) generate a response based on the first value, the activity type, activity intensity, the received input, or a combination thereof

In some embodiments, the one or more processors may include a first processor operably coupled to the mechanical blood pump. In some embodiments, the one or more processors may include a second processor operably coupled to the first processor via a network.

In some embodiments, generating a response may include causing an adjustment of a flow rate of the blood pump based on the determined value representative of cardiac recovery.

In some embodiments, generating a response may include generating an alert based on, e.g., the determined value representative of cardiac recovery, the activity type, activity intensity, the received input, or a combination thereof. In some embodiments, the alert may be sent to a processor on a device associated with the subject, to a processor on a device associated with a medical practitioner, and/or to emergency services personnel. In some embodiments, the alert may be sent to a predefined person or group of people.

In some embodiments, generating an alert may include identifying a potential issue based on the first value, the activity type, activity intensity, the received input, or a combination thereof. In some embodiments, the alert (which may have been sent to one or more individuals) may include the potential issue. In some embodiments, the alert may include a location of the subject.

In some embodiments, the one or more processors may be further configured to determine a location based on the received input and store the location. In some embodiments, the stored locations may be configured to be accessed by a clinician and/or researcher.

In some embodiments, the mechanical blood pump may be inserted into a chamber of a subject's heart.

In some embodiments, the one or more sensors may include an accelerometer, a gyroscope, a heart rate sensor, and a pressure sensor. In some embodiments, the sensor(s) include a sensor disposed in or on the mechanical blood pump. In some embodiments, the sensor(s) include a sensor disposed in or on a patch operably coupled to the subject. In some embodiments, the sensor(s) include a sensor disposed in or on a wearable device operably coupled to the subject.

In some embodiments, determining the activity type may include determining if a subject is engaged in a specific activity. In some embodiments, the specific activities may include walking, sitting, standing, lying down, and changing from lying down to sitting up. In some embodiments, a machine learning algorithm is used to determine the activity type and the activity intensity.

In some embodiments, a method for monitoring and supporting mechanical blood pumps in use with a subject may be provided.

The method may include (i) receiving input from the one or more sensors; (ii) determining an activity type, an activity intensity, or both an activity type and intensity based on the input; (iii) determining a first value representative of cardiac recovery based on the input and the determined activity type, intensity, or both type and intensity; and (iv) generating a response based on the first value, the activity type, activity intensity, the received input, or a combination thereof.

In some embodiments, the method may include generating a response includes causing an adjustment of a flow rate of the blood pump based on the determined value representative of cardiac recovery.

In some embodiments, the method may include generating a response includes generating an alert based on the determined value representative of cardiac recovery, the activity type, activity intensity, the received input, or a combination thereof. In some embodiments, the alert may be sent to a processor on a device associated with the subject, to a processor on a device associated with a medical practitioner, and/or to emergency services personnel. In some embodiments, the alert may be sent to a predefined person or group of people.

In some embodiments, generating an alert may include identifying a potential issue based on the first value, the activity type, activity intensity, the received input, or a combination thereof.

In some embodiments, the alert (which may be sent to one or more individuals) may include the potential issue. In some embodiments, the alert may include a location of the subject.

In some embodiments, the method may include determining a location based on the received input and storing the location. In some embodiments, the stored locations may be configured to be accessed by a clinician and/or researcher.

In some embodiments, the mechanical blood pump may be inserted into a chamber of a subject's heart.

In some embodiments, the one or more sensors may include an accelerometer, a gyroscope, a heart rate sensor, and a pressure sensor. In some embodiments, the sensor(s) include a sensor disposed in or on the mechanical blood pump. In some embodiments, the sensor(s) include a sensor disposed in or on a patch operably coupled to the subject. In some embodiments, the sensor(s) include a sensor disposed in or on a wearable device operably coupled to the subject.

In some embodiments, determining the activity type may include determining if a subject is engaged in a specific activity. In some embodiments, the specific activities may include walking, sitting, standing, lying down, and changing from lying down to sitting up. In some embodiments, a machine learning algorithm is used to determine the activity type and the activity intensity.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of one embodiment of a system for modulating support of a mechanical blood pump.

FIG. 2 is an illustration of one embodiment of an intravascular heart pump system located in a heart.

FIG. 3 is a cross-sectional side view of an embodiment of a blood pump.

FIG. 4 is a flowchart of an embodiment of a disclosed method.

FIG. 5 is a flowchart of an embodiment of a disclosed method.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others.

The inventors have recognized that there are numerous benefits that can accrue from a support device that can provide real-time, quantitative, continuous, direct measurements useful for estimating cardiac function.

Traditionally, left-ventricular pressure (LVP) is estimated by measurement of a Pulmonary Arterial Wedge Pressure (PAWP) or Pulmonary Capillary Wedge Pressure (PCWP) in which a pulmonary catheter including a balloon is inserted into a pulmonary arterial branch. PAWP and PCWP may not be an effective measurement of cardiac health, as the pulmonary arterial catheters are intermittent, indirect, and inconsistent, resulting in incorrect data which cannot be used reliably by clinicians to make clinical decisions regarding the level of cardiac support required by a subject.

Further, such qualitative measurements and periodic decisions may not provide real-time alterations to support levels based on the activities actively undertaken by a subject. For example, a subject may need different levels of support when the subject is sitting, versus when the subject moves from a prone position to a sitting position, versus when the subject attempts to stand up, walk, etc.

Recovery is presently assessed only in the clinical setting at prescribed follow-up intervals. For example, things like walk test and ejection fraction are typically used to assess at the predetermined time points. Further, existing physiological control for device support modulation has primarily been conducted academically and pre-clinically. These systems typically rely upon the generation of a physiological model (i.e., Windkessel), and the assessment of HR as an analog for activity level. The inventors have appreciated that there is no direct measure of activity and as a heart recovers, and that it is unknown if a particular physiologic response is a result of high intensity with moderate cardiac function or poor cardiac function with moderate intensity.

Among other benefits, the disclosed systems and method may alleviate such deficiencies.

To provide an overall understanding of the systems, method, and devices describe herein, certain illustrative embodiments will be described. Although the embodiments and features described herein are specifically described for use in connection with a percutaneous blood pump system, it will be understood that all the components and other features outlined below may be combined with one another in any suitable manner and may be adapted and applied to other types of cardiac therapy and cardiac assist devices, including cardiac assist devices implanted using a surgical incision, and the like. The disclosed systems and methods can be used by themselves, or in conjunction with other data systems for modulating the support provided by a blood pump to accommodate the physical activity of a subject and improve the subject's quality of life.

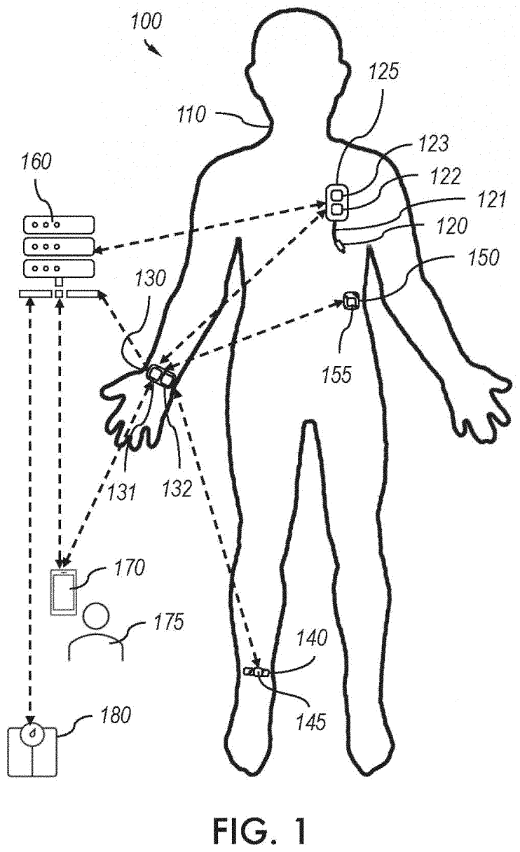

Various systems may be described with reference to FIG. 1. In FIG. 1, an embodiment of a system for modulating support of a mechanical circulatory support device (e.g., blood pump) 100 may be seen relative to a subject 110. The system may include a mechanical blood pump 120. The blood pump may be attached to or within a catheter 121, and may be implantable or insertable into a subject, such as into a subject's heart (not shown). The blood pump may include a motor and a rotor configured with blades to move blood from one portion of the vascular system to another.

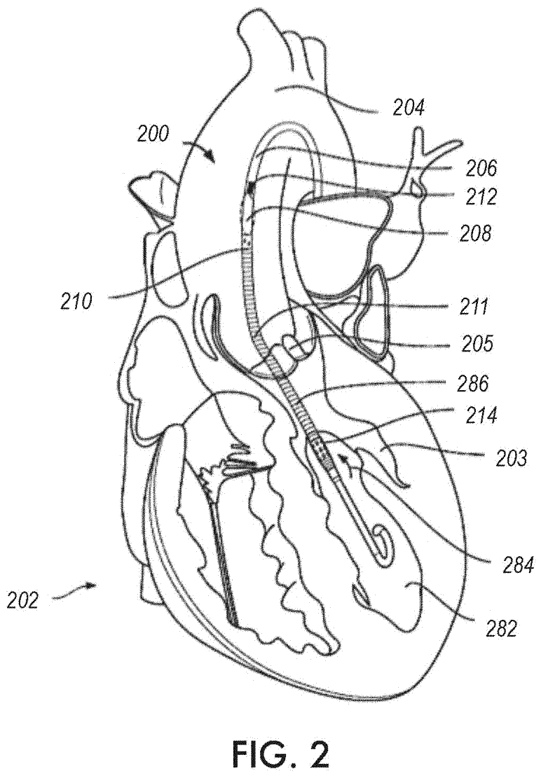

Referring to FIG. 2, an embodiment of such a configuration is detailed. A cardiac assist device, such as an intravascular blood pump 200, may be positioned in a blood vessel of a subject, such as in a heart 202. In FIG. 2, a left ventricle 203, aorta 204, and aortic valve 205 are seen. The intravascular blood pump 200 may include a catheter 206, a motor 208, a pump outlet 210, a cannula 211, a pump inlet 214, and a pressure sensor 212.

The motor may be configured to cause a rotor (not shown in FIG. 2) to rotate, resulting in blood flowing from the pump inlet 214 through the cannula 211 to the pump outlet 210. The positioning of the motor may vary. In some embodiments, the motor may be coupled to the rotor via an elongate mechanical transmission element, such as a flexible drive shaft, drive cable, or a fluidic coupling. In some embodiments, the motor may be positioned within a pump housing, proximal to the rotor. In some embodiments, the motor may be positioned proximal to the pump housing. The motor also may be positioned extracorporeally in some embodiments. In some embodiments, the motor may be operably coupled at its proximal end to the catheter, and at its distal end to the cannula.

In some embodiments, the cannula may be positioned across the aortic valve, such that the pump inlet may be located within a first area of the subject's body (e.g., a left ventricle) and the pump outlet may be located within a second area of the subject's body (e.g., the aorta). This example configuration would allow the intravascular heart pump system to pump blood from the left ventricle into the aorta to support cardiac output. In some embodiments, the intravascular heart pump system may pump blood from the left ventricle into the aorta in parallel with the native cardiac output of the heart.

The blood flow through a healthy heart is typically about 5 liters/minute. In some embodiments, the blood flow through the intravascular heart pump system may be adjusted to be a similar flow raw as compared to a healthy heart. In other embodiments, the blood through the intravascular heart pump system may be adjusted to be a different flow rate as compared to a healthy heart. For example, in some embodiments, the flow rate through the intravascular heart pump system may be 0.5 liters/minute, 1 liter/minute, 1.5 liters per minute, 2 liters/minute, 2.5 liters/minute, 3 liters/minute, 3.5 liters/minute, 4 liters/minute, 4.5 liters/minute, 5 liters/minute, greater than 5 liters/minute or any other suitable flow rate.

The motor of the intravascular heart pump system may vary in any number of ways. For example, the motor may be an electric motor. In some embodiments, the motor may be operated at a constant rotational velocity to pump blood from the left ventricle to the aorta. Operating the motor at a constant velocity generally requires supplying the motor with varying amounts of current because the load on the motor varies during the different stages of the cardiac cycle of the heart. For example, when the mass flow rate of blood through the blood pump into the aorta increases (e.g., during systole), the current required to operate the motor increases. In some embodiments, this change in motor current may be used to help characterize cardiac function. Detection of mass flow rate using motor current may be facilitated by the position of the motor, which is aligned with the natural direction of blood flow, e.g., from the left ventricle into the aorta.

Detection of mass flow rate using motor current may also be facilitated by the small size and/or low torque of the motor. In some embodiments, the motor may have a diameter of, e.g., about 4 mm, but any suitable motor diameter may be used provided that the rotor-motor mass is small enough, has low enough torque, and is positioned such that it is able to respond to changes quickly and easily in the physiologic pressure gradient across the pump. In some implementations, the diameter of the motor may be less than 4 mm. In some implementations, the diameter of the motor may be less than 3.5 mm.

In some implementations, one or more motor parameters other than current, such as power delivered to the motor, are measured. In some implementations, the motor may operate at a constant velocity. In some implementations, the speed of the motor may be varied over time (e.g., as a delta, step, sinusoid, and/or ramp function) to probe the native heart function. In some embodiments, the variation over time may be constant (e.g., a simple sinusoidal variation), and/or the deltas, steps, or ramps may involve regular changes (e.g., a fixed 1000 rpm change every 5 seconds, or a constant ramp to increase rotational speed by 5000 rpm over 1 minute). In some embodiments, the variation over time may not constant (e.g., a sinusoidal variation that may periodically change from a first frequency to a second frequency), and/or the deltas, steps, or ramps may involve irregular changes (e.g., a first change of 1000 rpm after 5 seconds, then a change of 400 rpm after 3 seconds).

The pressure sensor 212 of the intravascular heart pump system may be disposed at various locations on the pump, such as on the motor, or at the outflow of the pump, i.e., at a pump outlet 210. Placement of the pressure sensor at the pump outlet may enable the pressure sensor to measure the true aortic pressure (AoP) when the intravascular blood pump system is positioned across the aortic valve. In certain implementations, the pressure sensor of the intravascular heart pump may be disposed on the cannula, on the catheter, or in any other suitable location.

In some embodiments, the pressure sensor may detect blood pressure in the aorta when the intravascular heart pump system is properly positioned in the heart. The blood pressure information can be used to properly place the intravascular heart pump system in the heart. For example, the pressure sensor can be used to detect whether the pump outlet has passed through the aortic valve into the left ventricle which would only circulate blood within the left ventricle rather than transport blood from the left ventricle to the aorta.

In some implementations, the pressure sensor may be a fluid filled tube, a differential pressure sensor, hydraulic sensor, piezo-resistive strain gauge, optical interferometry sensor or other optical sensor, MEMS piezo-electric sensor, or any other suitable sensor.

The intravascular heart pump may be inserted in various ways, such as by percutaneous insertion into the heart. For example, the intravascular heart pump may be inserted through a femoral artery (not shown), through the aorta, across the aortic valve, and into the left ventricle. In certain implementations, the intravascular heart pump system may be surgically inserted into the heart (e.g., into a chamber of a subject's heart). In some implementations, the intravascular heart pump, or a similar system adapted for the right heart, may be inserted into the right heart. For example, a right heart pump similar to the intravascular heart pump shown in FIG. 2 may be inserted through the femoral vein and into the inferior vena cava, bypassing the right atrium and right ventricle, and extending into the pulmonary artery. Alternatively, a right heart pump can be inserted through the internal jugular vein and superior vena cava, and a left heart pump can be inserted through the axillary artery.

In certain implementations, the intravascular heart pump may be positioned for operation in the vascular system outside of the heart (e.g., in the aorta). By residing minimally invasively within the vascular system, the intravascular heart pump system is sufficiently sensitive to allow characterization of native cardiac function.

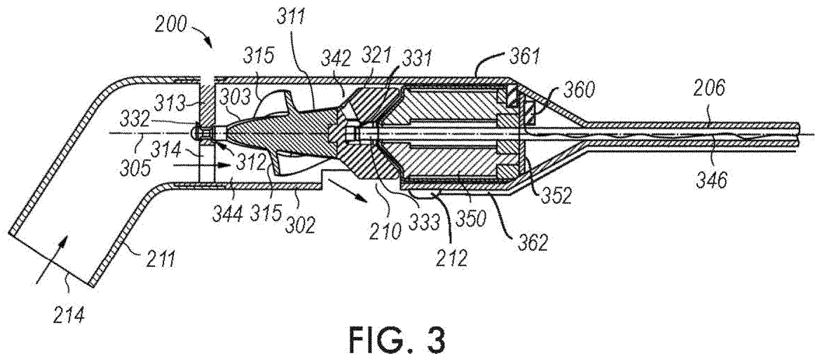

Referring to FIG. 3, a cross-sectional view of an embodiment of a blood pump 200 according to some embodiments is illustrated. The blood pump 200 may include the rotor 311 as described above, and an electric drive unit 350 (which may be, e.g., the pump motor, and may include a stator). The blood pump may include a pump casing 302 with a blood flow inlet 214 and a blood flow outlet 210. The blood pump may be designed as an intravascular pump, also called a catheter pump, and may be deployed into a patient's blood vessel by means of a catheter 206. In some embodiments, the blood flow inlet 214 may be at the end of a flexible cannula 211 which may be placed through a heart valve, such as the aortic valve, during use. The blood flow outlet 210 may be located in a side surface of the pump casing and may be placed in a heart vessel, such as the aorta. The blood pump may be electrically connected with electric line(s) 346 extending through the catheter. The electrical line(s) may be used for, e.g., supplying the blood pump with electric power in order to drive the blood pump by means of electric drive unit 350, and/or communicating to or from sensor(s) in or on the blood pump.

If the blood pump is intended to be used in long term applications, i.e., in situations in which the blood pump may be implanted into the patient for several weeks or even months, electric power may be supplied by means of a battery. This may allow a patient to be mobile because the patient is not connected to a base station by means of cables. The battery can be carried by the patient and may supply electric energy to the blood pump, e.g., wirelessly.

The blood may be conveyed along a passage 344 connecting the blood flow inlet 214 and the blood flow outlet 210 (blood flow indicated by arrows). Rotor 311 (also referred to as an impeller) as described above may be provided for conveying blood along the passage 344. In some embodiments, the rotor may be mounted to be rotatable about an axis of rotation 305 within the pump casing 302 by means of a first bearing 331 and a second bearing 332. The axis of rotation 305 may be along the longitudinal axis of the rotor 311. Both bearings 331, 332 may be contact-type bearings in this embodiment. At least one of the bearings 331, 332 may be a non-contact-type bearing, however, such as a magnetic or hydrodynamic bearing. The first bearing 331 may be a pivot bearing having spherical bearing surfaces that allow for rotational movement as well as pivoting movement to some degree. A pin 333 may be provided, forming one of the bearing surfaces. The second bearing 332 may be disposed in a supporting member 313 to stabilize the rotation of the rotor, the supporting member having at least one opening 314 for the blood flow. Blades 315 may be provided on the rotor for conveying blood once the rotor rotates. Rotation of the rotor may be caused by the drive unit 350, which may be magnetically coupled to a magnet 321 at the proximal end of rotor 311.

It will be understood that the illustrated blood pump is a mixed-type blood pump, with the major direction of flow being axial. It will be appreciated that the blood pump could also be a purely axial blood pump, depending on the arrangement of the rotor, and in particular the blades.

Skilled artisans will recognize how to configure an electric drive unit to be capable of magnetically interacting with said intravascular blood pump rotor. In some embodiments, the electric drive unit should be configured to be adjacent to, but physically separated from, the rotor.

The blood pump may include one or more sensors. In some embodiments, a single sensor may be incorporated. In some embodiments, a plurality of sensors may be included. In some embodiments, wherein the one or more sensors comprises a sensor in or on the mechanical blood pump. In some embodiments, one or more sensors may be positioned at a pump outlet. In FIG. 3, a pressure sensor 212 is shown as positioned on an external surface 361 of the pump casing 302 near the at the pump outlet 210. In some embodiments, one or more sensors 360 may be present within the pump casing. In FIG. 3, the sensor is shown as being configured to communicate via signals through a lumen 362. In FIG. 3, the lumen is shown to be external to the catheter and the pump casing, however, in some embodiments, the lumen may be internal to some or all of the pump casing and/or catheter.

Also seen is in FIG. 3 is a second sensor 360. In some embodiments, the second sensor may be coupled to, e.g., a proximal end 352 of the electric drive unit, and may be positioned within the pump casing. The second sensor may communicate via the electric line(s) 346. As will be understood, the sensor may also include, e.g., a printed circuit board (not shown), and some or all the circuitry needed by the sensor.

Referring again to FIG. 1, the blood pump may be operably controlled by one or more processors 122, which may be operably communicating with one or more other components 123, such as wired and/or wireless communication interfaces, one or more sensors, a memory, a non-transitory computer-readable storage medium, etc. The one or more processors may optionally be located within a housing 125. The housing may be operably coupled to the catheter. In some embodiments, the housing may be directly coupled to the catheter 121. In some embodiments, the housing may be indirectly coupled to the catheter. In some embodiments, the housing may be removable. In some embodiments, the housing may have a door, port, or hatch that allows the internal components within the housing to be accessed.

In the system 100 shown in FIG. 1, the blood pump may be configured to operably communicate with one or more processors. The one or more processors may comprise the one or more processors 122 in the optional housing, and/or may be one or more processors present in a separate controller 130 (shown here worn on a subject's wrist), or in a remote location, such as a computer, mobile device, or remote server 160. The location of the controller may vary in other embodiments, as will be appreciated. For example, in some embodiments, the controller may be worn on a wrist, around and arm, a leg, a waist (including, e.g., on a belt), and/or around a neck, to name a few. In some embodiments, the controller may not be worn by the subject, but instead may be on another suitable device, such as on a moveable cart, on a wheelchair, and/or on another device (e.g., moveable device). In some embodiments, the controller may be a mobile phone or tablet.

In FIG. 1, a controller 130 is shown as a separate wearable device comprising one or more processors 131 and one or more other components 132, such as wired and/or wireless communication interfaces, sensors, memory, and/or a non-transitory computer-readable storage medium. In some embodiments, the one or more processors 131 of the controller may be operably communicating with the blood pump, either wired or wirelessly. In some embodiments, the one or more processors 131 may also be operably communicating with other sensors or devices, such as other sensors, including, e.g., a wearable device 140 (such as a watch, ring, etc.) and/or a substrate 150 (such as a patch). In some embodiments, the one or more processors may operably communicate with a remote server 160. In some embodiments, the remote server may, as an alternate or additional approach, communicate directly with the blood pump. All such communications between components may independently be wired or wireless communication and may independently be bidirectional or monodirectional.

In some embodiments, the one or more processor(s) in the controller may communicate with one or more processor(s) on a remote device (such as a remote server, a laptop, tablet, or mobile device) over a network.

The system described herein may contain a plurality of sensors, each of which may be configured to provide at least one of two types of information: (i) information related to the motion or position of the body or other non-cardiac related information of the patient, and (ii) information related to the recovery of cardiac function. In some embodiments, the plurality of sensors includes a sensor in or on the pump and may include a sensor in or on a wearable device operably coupled to the subject. In some embodiments, the plurality of sensors includes a sensor in or on the pump and may include a sensor in or on a patch operably coupled to the subject.

In some embodiments, the information related to the motion or position of the body may be captured by one or more single or multi-axis gyroscope(s) and/or accelerometer(s). In some embodiments, the system may contain two or more of these sensors, on different portions of the body. For example, the embodiment in FIG. 1 may utilize at least one sensor on a first extremity of the subject (e.g., one of the other components 132 in a device worn on the wrist is a sensor), and at least one sensor on a second extremity of the subject (e.g., sensor 145 operably coupled to a wearable device 140 (or substrate) that is attached to the foot and/or leg of the subject).

In some embodiments, other types of sensors, besides gyroscopes and accelerometers, may be utilized as appropriate. In some embodiments, a plurality of physiological parameters is determined. In some embodiments, a plurality of physiological parameters, such as SpO2, pulse rate, and/or ejection time, may be monitored, either directly via sensors, or derived from sensor data. In some embodiments, the information related to the recovery of the cardiac function may be captured by one or more sensors configured to capture heart rate, blood oxygen saturation (SpO2), a pressure (such as blood pressure, left ventricular pressure, left ventricular end diastolic pressure, etc.), or a flow rate or velocity (such as blood volumetric rate or blood velocity, e.g., within a vein, etc.). In some embodiments, environmental sensors may be incorporated. In some embodiments, the sensor data may include temperature and/or humidity data.

Various sensor types may be utilized as appropriate. In some embodiments, a strain sensor 155, which may be attached to a substrate 150, may be utilized, such as in a subject's abdominal region to aid in detecting when a subject is bending. In some embodiments, one or more accelerometers may be coupled to the pump. In some embodiments, one or more accelerometers may be coupled to a wearable device and/or patch coupled to the user. In some embodiments, a sensor may be used for multiple purposes. For example, in some embodiments, an optical sensor may be used as a vibration sensor.

In some embodiments, the system may include one or more remote devices 170, 180 (see FIG. 1), such as a mobile phone, laptop, or tablet, or medical device (including, e.g., a scale, thermometer, etc.).

In some embodiments, the one or more processors may require other information beyond what the sensors in or on the patient's body can provide. In some embodiments, for example, remote devices (such as the one or more remote devices 170, 180) may be able to provide such information, especially for data that may not be capable of varying significantly over the course of a few minutes, hours, or days. For example, the one or more processors may also require a subject's height and/or weight. A subject's height may be measured once, and transmitted to the one or more processors separately, e.g., as a data packet from a remote device 170, which may be a remote computing device such as phone, tablet, desktop computer, laptop computer, etc., where a user (such as a first user 175, such as a practitioner), associated with the computing device, has entered the information (here, the subject's height) into the device, and that information is then transmitted to the controller and/or a remote server.

Similarly, a subject's weight may be monitored on an infrequent basis (e.g., daily, weekly, etc.) and provided to the one or more processors in a similar fashion. Alternatively, the one or more processors may receive some or all of this additional data automatically from an appropriate device, e.g., a weight from a sensor in a remote device 180 (here, shown as a scale).

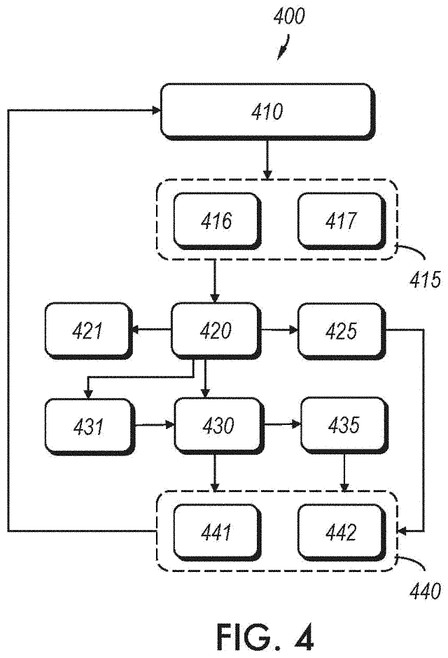

In some embodiments, the components may be utilized in a variety of ways, based on a desired outcome. Referring to FIG. 4, an embodiment of a method may be provided. The method 400 may include receiving 410 inputs from one or more sensors, such as those disclosed herein.

In some embodiments, the method may include preprocessing 415 the received input. In some embodiments, this may include time stamping and storing 416 all received data.

In some embodiments, this may include extracting 417 one or more features from the received data. For example, a “gait” feature may be extracted from accelerometer data. In some embodiments, this extraction process may require transformation of the sensor data to a different domain, such as the frequency domain. In some embodiments, the extraction process may require that a filter, such as a low-pass filter, be applied to the sensor data or the transformed data. The “gait” feature may appear as the dominant frequency at, for example, up to 1 Hz, up to 2 Hz, up to 3 Hz, up to 4 Hz, or up to 5 Hz.

In some embodiments, the method may include determining 420 an activity type, an activity intensity, or both an activity type and intensity based on the received input. More specifically, using the received data, including the data provided by the various sensors, the one or more processors can classify or determine what activity is occurring based on sensor readings collected over a period of time. In some embodiments, only the sensors related to the motion or position of the body may be utilized for this classification. In some embodiments, determining the activity type may include determining if a subject is engaged in a specific activity, such as walking, sitting, standing, lying down, and/or changing from lying down to sitting up. For example, in some embodiments, an algorithm can be trained to receive the gyroscope and accelerometer data from sensors on forearm and sensors on the lower leg, and determine if the subject is running, walking, sitting, trying to sit up, standing, or lying down.

The algorithm may determine the activity type at any time and may make additional determinations at regular and/or irregular intervals. In some embodiments, the classifier may continuously determine the activity type. In some embodiments, the classifier determines the activity type at regular intervals, such as every 30 seconds, or every minute.

In some embodiments, the classifier may consider data only from sensor data acquired in a most recent period of time (tₚ) prior to the current time (t) (e.g., where t−tₚ is ≤1 minute, ≤2 minutes, or ≤5 minutes) when determining what type of activity is currently being performed by the subject.

In some embodiments, the period of time the classifier considers data may be greater than the interval between when the classifier attempts to determine the activity type. For example, in some embodiments, the classifier may be configured to determine, every 30 seconds, what activity the subject is performing, based on the previous 5 minutes of sensor data.

In some embodiments, the method may include time stamping and storing 421 the determined activity type. The classifier may also utilize this time stamped activity type information.

In some embodiments, the time stamped activity type information can be used to predict timing of certain activities, or groups of activities that are likely to occur in sequences.

There are many types of algorithms and/or classifiers known in the art; any appropriate algorithm or classifier may be utilized for these determination/classification steps. In some embodiments, a supervised machine learning algorithm may be utilized, such a k-nearest neighbor (KNN) algorithm.

In some embodiments, it may not be necessary to determine an exact activity being undertaken. That is, it may not always be necessary to determine whether the subject is brushing teeth or taking a shower. Instead, it may only be necessary to simply classify the activity as a general type or category. In some embodiments, it may only be necessary to know the general orientation of a subject's chest (e.g., substantially vertical, substantially horizontal, or reclined). In other embodiments, it may be useful to classify or categorize activities by the expected stress levels on the heart; that is, certain types of positions or motions would be considered “low” stress, others “medium” stress, and still others “high stress” for a given subject at a given level of recovery.

In some embodiments, the activity or classification of the activity may be based on the speed or rate of movement. For example, if the subject is detected as being in a sitting position but is moving at a rate that is akin to a normal walking pace, the algorithm may determine that the subject is being pushed in a wheelchair. If the subject is detected as sitting, but the arms are moving in a regular pattern, and the subject's body is moving at a slow pace, the algorithm may determine that the subject is moving themselves in a wheelchair.

In addition to the type of activity, or as an alternative to the type, the intensity of the activity may be determined.

The intensity of the activity may be based on physiological sensors, either alone or in combination with the motion sensors and/or the determined type of activity being performed. For example, in some embodiments, a heart rate sensor may be used, in combination with the subject's age, to determine a heart rate zone. For example, with a maximum heart rate (HR_{MAX}) estimated at as HR_{MAX}=220-age (or 205.8−(0.685×age), or some other appropriate correlation), in some embodiments, the intensity could be a percentage of HR_{MAX}. In other embodiments, the intensity could be a descriptive range; a “low” intensity could be <50% of HR_{MAX}, a “moderate” intensity could be 50-85% of HR_{MAX}, a “high” intensity could be >85% of HR_{MAX}.

Still other embodiments may determine intensity based in part changes in SpO₂ while the activity is occurring. For example, if sitting up leaves a subject gasping for breath, the SpO₂ readings will reflect that as a drop in SpO₂. In such an example, the greater the drop, the more intense the activity.

In some embodiments, the one or more processors may be configured with a trained machine learning algorithm to gather the sensor data and classify the intensity of the activity.

In some embodiments, the type and intensity of the activity are determined at substantially the same time. For example, a trained machine learning algorithm can be utilized to classify the sensor data as indicating a person is performing a low intensity walk or a high intensity walk.

In some embodiments, the method may include determining 425, based on the received input and the determined activity type and/or intensity, if there is motion present that is abnormal for a given activity type and/or intensity. For instance, if the general activity type is determined to be “changing position from laying down to sitting up”, and the sensors also indicate sudden downward acceleration of the blood pump, the method may detect an indication of abnormal behavior, since it would be expected that any acceleration would be upward, as the chest of the subject lifts from the bed. Instead, in this example, a sudden downward acceleration may be an indication the subject has fallen. In some embodiments, this step may include making a determination as to the type of abnormal motion that is detected (e.g., a fall or slip, a change in pump placement, etc.)

In some embodiments, the method may include determining 430 a value representative of cardiac recovery based on the received input and the determined activity type, intensity, or both type and intensity. That is, the transient assessment of physiological response, under different activity types, intensities, and durations, can be used as an indicator of cardiovascular recovery.

In one embodiment, the method may include determining 431 one or more trends in heart rate and cardiovascular-related pressures, such as left-ventricular diastolic filling pressure and/or systolic pressure gradient, over time while specific activities are being performed, or after specific activities were performed. In such embodiments, the determining 430 of a cardiac recovery value may be further based on such determined trends.

In some embodiments, the method may include determining 435 whether the activity type, intensity, or both are desirable based on the value representative of cardiac recovery. For example, immediately after surgery, it is understood that a subject should most likely not be running down hallways, and if they are, the physicians should be made aware of that fact. In another example, if a recovering subject is running at a high intensity but the subject's cardiac recovery value is low, there may be a determination that the intensity is not desirable given the current cardiac recovery value. This determination may be based on, e.g., curves saved on a database or formulas defined for a particular activity, etc. For example, a database operably coupled to the one or more processors making this determination that may include a formula for one or more given activities. Such formulas may define a desirable maximum intensity of 0 (i.e., the activity is not desirable) if cardiac recovery is below a certain value (e.g., recovery less than 0.5), and then a defined desirable maximum intensity that ramps up linearly (or non-linearly) from 0 to 1 as cardiac recover value increases (e.g., here, from 0.5 to 1).

In some embodiments, the method may also include generating 440 a response. In some embodiments, the generated response may be based on the value representative of cardiac recovery, the determined activity, the determined intensity, and/or the received input. In some embodiments, the generated response may be based on a determined issue. In some embodiments, the issue may be, e.g., a determined abnormal motion, or a determination that the activity is not desirable given the cardiac recovery as disclosed herein.

In some embodiments, generating a response may include causing 441 an adjustment of a flow rate of the blood pump, which may be, e.g., based on the determined value representative of cardiac recovery.

For example, once a cardiac recovery parameter has been determined, the parameter can be used to determine if an adjustment of a flow rate of the blood pump is necessary, and if so, by what amount, at what rate of change, and/or for how long. In some embodiments, the cardiac recovery parameter is a value between 0 and 1 (with 0 being zero recovery, and 1 being full or sufficient recovery). In one embodiment, the system calculates the increase in motor speed as: k(1-cardiac recovery parameter), where k is a weighting factor based on the activity type and intensity.

It will be understood that in some embodiments, adjustments may sometimes be tied to a patient's cardiac recovery level by itself, adjustments may sometimes be tied to the cardiac recovery level and other factors, and some adjustments may not be tied to cardiac recovery levels at all. For example, in some embodiments, the system may simply detect if additional support is needed, based one or more physiological parameters (e.g., blood pressure and/or heart rate), and/or activity type, and/or intensity.

In some embodiments, there may be two or more domains where it may be advantageous to set the support value provided by the system. For example, it may be useful to have at least two support levels, a first being a resting support based on cardiac recovery, and a second being an active support based on activity intensity, type and cardiac recovery.

As an example, adjustments could be made as part of an effort to wean a patient off the level of support being offered by the pump. This can be considered a technique for “training” the heart for recovery and slowly reduce the amount of support such that the native heart can pick up the slack and get stronger over time. In some embodiments, this could be done by adjusting the baseline level of support (e.g., a minimum pump speed) provided by the device over time and to monitor the heart during such adjustments. In some embodiments, the adjustments could be based on how the heart is recovering over time (e.g., continuing to reduce the level of patient support when a noted improvement in heart recovery is recognized. In some embodiments, the level of support could be provided on a continuous downward slope, as the level of recovery changes from 0 to 100%. In some embodiments, the cardiac recovery parameter may be a value between 0 and 1 (with 0 being zero recovery, and 1 being full or sufficient recovery). In some embodiments, the slope may be linear. In other embodiments, the slope may be non-linear. In one embodiment, the system may calculate the baseline support motor speed (b) as: b=m+z(1-cardiac recovery parameter), where m is a predetermined minimum motor speed and z is a predetermined scaling factor. Clearly, here, the adjustment in speed may be z times the change in cardiac recovery parameter. In some embodiments, the level of support could be provided as a plurality of step changes as the level of recovery changes from 0 to 100%. In one embodiment, the system may set the baseline support motor speed as m+z (when recover is <25%), m+0.67z (when recovery is <50%), m+0.33z (when recovery is <75%), and m (when recovery ≥75%). In such an embodiment, the step adjustments could be made as the determined recovery changes.

As another example, and optionally in addition to the above-described baseline support adjustments, adjustments could be made to provide an additional, or alternative, recovery metric. For example, in hospital settings, a patient using a blood pump may be asked to perform a 6-minute walk test, where the blood pump is reduced to the lowest support levels possible, and the patient's cardiac support is measured as they walk, with the distance they walk being used as a metric related to recovery. Here, adjustments or modulations in speed could be made to provide an alternative to the 6-minute walk test. For example, in one embodiment, physiological parameters may be monitored before and after a change in speed, while the patient is performing a target activity (e.g., walking, resting, or even lying down), which may originally have been at the patient's baseline level of support. In such an example, the differences in the physiological parameters may then be used to determine a recovery value. In some embodiments, rates of change of the physiological parameters after a change are also considered when determining recovery rate. In some embodiments, the maximum differences are considered. In some embodiments, the minimum differences are considered. For example, if at a point in time after a change, the system measures heart rate, and the heart rate is initially 20 bpm faster than the pre-change heart rate, but then eventually the heart rate slows to the pre-change heart rate, that would be a maximum difference of 20 bpm and a minimum difference of zero. In some embodiments, a single step change decrease in motor speed is used for the test, after which the speed is increased back to its original level. In some embodiments, the decrease is a continuous change from its original setting to a decreased level, then back to the original setting. The maximum decrease in speed from the baseline setting may be a fixed amount or may be a percentage of the baseline speed. For example, in one embodiment, the system may monitor blood pressure and heart rate while the patient is walking.

The system may then drop the speed from its baseline support speed (b) to a reduced speed (r) that is 80% of b, and continues monitoring blood pressure and heart rate.

As another example, and optionally in addition to ongoing baseline support adjustments, adjustments could be made to “boost” a patient's support in acute situations. That is, in some embodiments, even if you are “training” the heart to wean it off support, it may be advantageous to want that same patient to get additional support when they are performing certain activities (e.g., for improved quality of life and/or to encourage heart recovery). For example, in some embodiments, the system may determine the patient is attempting to walk up a flight of stairs. In this example, the system could provide a short “boost” of support based on the determined activity. As will be appreciated, in some embodiments, the determined “boost” may be determined based on the herein described method of determining a value representative of cardiac recovery based on input received from one or more sensor and the activity type, intensity, or both type and intensity (e.g., when the patient is walking up the stairs.

In some embodiments, the system may make a predetermined adjustment (increase) to the baseline support motor speed based on the determined activity type (e.g., motor speed=b+y, where y is the speed adjustment based on the activity type). In some embodiments, y may be a predetermined fixed value (for example, +5,000 rpm). In some embodiments, y may be based on the baseline motor speed (for example, +5% of baseline motor speed).

In still other embodiments, an adjustment may be made based only on a level of intensity of an activity. For example, in some embodiments, it may be determined that a recovering patient is walking, and it is determined that while walking, the intensity has changed from “high” intensity to “medium” intensity. In this example, the system may be configured to decrease speed by a fixed (e.g., −5000 rpm) or relative amount (e.g., −15% of baseline speed) based on the reduction in intensity. In some embodiments, the change may be a step change, or may be a continuous change from the current speed to the adjusted speed.

In yet other embodiments, an adjustment may be made based only on physiological parameters. For example, in some embodiments, it may be determined that—regardless of recovery levels—a change in support is needed for some reason (e.g., blood pressure too high or too low, etc.). In some embodiments, the system may be configured to make increase or decrease speeds based on the received input from various sensors.

In some embodiments, generating a response may include generating 442 an alert when abnormal movement for a given activity is detected and/or if a given activity and/or intensity is determined to be undesirable for a given degree of cardiac recovery. In some embodiments, the alert may be sent to a predefined person or group of people. In some embodiments, the alert may be sent to a processor on a device associated with the subject. In some embodiments, the alert may be sent to a processor on a device associated with a medical practitioner. In some embodiments, the alert may be sent to emergency services personnel.

In some embodiments, this may include generating a visual and/or auditory alert for a medical practitioner (e.g., doctor, nurse, etc.). For example, in some embodiments, the controller may send an alert to a remote device 170 associated with a user 175 (such as the medical practitioner), which may cause the remote device to provide a visual and/or auditory alert to the user. In some embodiments, the controller may include a speaker or display (see, e.g., other components 132 in FIG. 1). Such a speaker or display may issue a visual and/or auditory alert to the subject. In some embodiments, such alerts may include a warning. In some embodiments, such alerts may include actions to be taken.

In some embodiments, the controller may be configured to receive or determine a location (e.g., from a GPS chip, from triangulation of wireless signals, etc.), and the alert may include that location. Thus, in some embodiments, a medical practitioner (such as the subject's physician) may receive an alert indicating the patient's specific location (e.g., coordinates) or general location (at home, at hospital, etc.), and may include a warning (for example, “subject has likely experience a fall”), and/or may include actions to be taken (for example, “verify status of subject”, “contact subject to verify status”, or “resecure patch to chest”).

In some embodiments, the alerts and/or warnings may require the at least one processor to receive feedback from the user and/or subject (as appropriate) to indicate the alert was received and/or that actions have been undertaken.

In some embodiments, the controller may include a microphone (see, e.g., other components 132 in FIG. 1). With a speaker and microphone, the one or more processors (and/or one or more medical practitioners) may be able to communicate with the subject, which can be used as part of any recovery and/or response actions. As will be understood, in some embodiments, the controller may include a means of wireless communication with a remote server and/or user, such as over the internet and/or using cellular data.

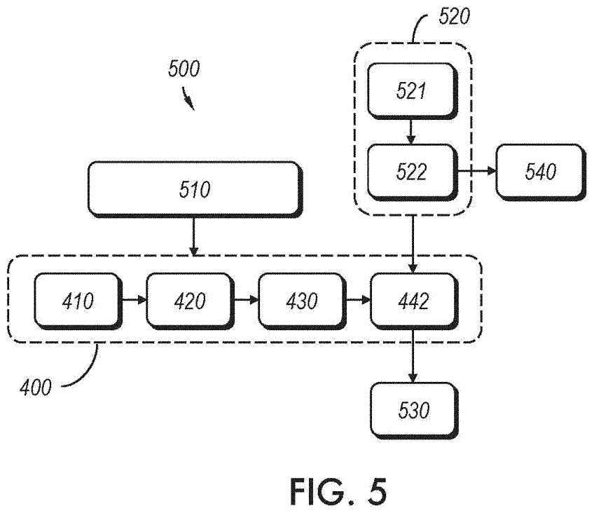

Referring to FIG. 5, in some embodiments, the method 500 may include pairing 510 an accessory (e.g., a mobile device such as a mobile phone, tablet, laptop, etc., a wearable such as a watch or ring, or a patch) with a controller. This may be done over any appropriate communications protocol. In some embodiments, this may include communicating using a standard communication protocol, such as, e.g., Bluetooth, WIFI, TCP/IP, etc.

The controller may then use data from the paired device(s) as described by the method 400 disclosed herein, including, e.g., receiving 410 the data and making determinations (e.g., determination 420 and determination 430) as disclosed herein based on the received data. As part of that method, and as disclosed herein, issues may be detected based on the received data, and alerts may be generated 442 based on those detected issues.

As disclosed herein, the issues may include, e.g., abnormal motions being detected, or the subject undertaking certain actions that may not be advisable for a given cardiac recovery value. Prior to generating the alert, the method may include determining 520 a location for use in the alert and/or who should receive the alert. For example, in some embodiments, the method may include receiving 521 data, e.g., entered in by a user (such as the subject or a medical practitioner). The data may include information related to who may be alerted (e.g., an emergency contact, a physician, emergency medical services, etc.). In some embodiments, the data may include information related to where the user is or what location data may be utilized. For example, in some embodiments, the user may enter in a particular location (such as a home address, a room in a hospital or recovery center, etc.), may indicate wireless location detection may be used, and/or may indicate GPS (or other location sensors) data may be used. Such information may be stored, e.g., in a database, such as on a remote server, or on the controller.

In some embodiments, the method may include identifying a current location 522. In some embodiments, this may include receiving GPS data or other location-related data (such as wireless signal strength) and may include converting that data into a specific location or specific area.

In some embodiments, the method may include the one or more processors receiving this information and then generating 442 the alert.

In some embodiments, generating the alert may include identifying a potential issue based on the first value, the activity type, activity intensity, the received input, or a combination thereof. One or more trained machine learning algorithms or lookup tables may be used to identify potential issue(s).

In some embodiments, generating the alert may include determining who should be notified, based on, e.g., the information configured by the user, etc. In some embodiments, determining who should be notified may also be based on the determined issue and data in a table that groups the potential issues into categories, where each category may indicate a different group of people should be identified, and/or the urgency of such a notification. For example, if the issue is a positive one, such as that a subject is recovering faster than normal, an alert may be generated that may send an email to the subject's physician. Conversely, if the issue is that the subject may have fallen and is currently non-responsive, emergency services may be contacted.

In some embodiments, the method may include storing 530 alerts and responses, as well as and received data that was used to generate the alert or response. In some embodiments, such stored data may be sanitized to remove user-identifiable data. In some embodiments, such storing includes storing a time from about the time the blood pump was inserted until the time of the alert.

In some embodiments, such as for clinical or safety reasons, the method may include monitoring and/or storing 540 the location data of one or more subjects, for some or all of the time that the blood pump is in use. In some embodiments, the stored locations are configured to be accessed by a clinician and/or researcher. In some embodiments, the stored locations are configured to be accessed by a physician.

Those skilled in the art will recognize or be able to ascertain using no more than routine experimentation, many equivalents to the specific embodiments of the invention described herein. Such equivalents are intended to be encompassed by the following claims.

What is claimed is:

1. A system for monitoring and supporting mechanical blood pumps in use with a subject, comprising:
a mechanical blood pump;
one or more sensors configured to detect a movement and a physiological condition of the subject; and
one or more processors configured to:
receive input from the one or more sensors;
determine an activity type, an activity intensity, or both an activity type and intensity based on the received input;
determine a first value representative of cardiac recovery based on the input and the activity type, intensity, or both type and intensity; and
generate a response based on the first value, the activity type, activity intensity, the received input, or a combination thereof

2. The system according to claim 1, wherein the one or more processors includes a first processor operably coupled to the mechanical blood pump.

3. The system according to claim 2, wherein the one or more processors includes a second processor operably coupled to the first processor via a network.

4. The system according to claim 1, wherein generating a response includes causing an adjustment of a flow rate of the blood pump based on the determined value representative of cardiac recovery.

5. The system according to claim 1, wherein generating a response includes generating an alert based on the determined value representative of cardiac recovery, the activity type, activity intensity, the received input, or a combination thereof.

6. The system according to claim 5, wherein the alert is sent to a processor on a device associated with the subject.

7. The system according to claim 5, wherein the alert is sent to a processor on a device associated with a medical practitioner.

8. The system according to claim 5, wherein the alert is sent to emergency services personnel.

9. The system according to claim 5, wherein the alert is sent to a predefined person or group of people.

10. The system according to claim 5, wherein generating an alert includes identifying a potential issue based on the first value, the activity type, activity intensity, the received input, or a combination thereof.

11. The system according to claim 10, wherein the alert includes the potential issue.

12. The system according to claim 5, wherein the alert includes a location of the subject.

13. The system according to claim 1, where in the one or more processors are further configured to determine a location based on the received input and store the location.

14. The system according to claim 13, wherein the stored locations are configured to be accessed by a clinician and/or researcher.

15. The system according to claim 1, wherein the mechanical blood pump is inserted into a chamber of a subject's heart.

16. The system according to claim 1, wherein the one or more sensors comprises an accelerometer, a gyroscope, a heart rate sensor, and a pressure sensor.

17. The system according to claim 1, wherein the one or more sensors comprises a sensor in or on the mechanical blood pump.

18. The system according to claim 17, wherein the one or more sensors further comprises a sensor in or on a patch operably coupled to the subject.

19. The system according to claim 17, wherein the one or more sensors further comprises a sensor in a wearable device operably coupled to the subject.

20. The system according to claim 1, wherein determining the activity type comprises determining if a subject is engaged in a specific activity, the specific activities comprising walking, sitting, standing, lying down, and changing from lying down to sitting up.

21. The system according to claim 1, wherein a machine learning algorithm is used to determine the activity type and the activity intensity.

22. The system according to claim 1, wherein the response includes adjusting a level of support, and wherein the one or more processors are further configured to monitor a parameter of heart recovery in response to adjusting the level of support.

23. The system according to claim 22, wherein the one or more processors are further configured to generate a second response based on the monitored parameter of heart recovery.

24. The system according to claim 23, wherein generating the second response includes adjusting a flow rate of the blood pump based on the monitored parameter of heart recovery.

25. A method for monitoring and supporting mechanical blood pumps in use with a subject, comprising:
receiving input from one or more sensors;
determining an activity type, an activity intensity, or both an activity type and intensity based on the received input;
determining a first value representative of cardiac recovery based on the received input and the determined activity type, intensity, or both type and intensity; and
generating a response based on the first value, the activity type, activity intensity, the received input, or a combination thereof

26. The method according to claim 25, wherein generating a response includes causing an adjustment of a flow rate of the blood pump based on the determined value representative of cardiac recovery.

27. The method according to claim 25, wherein generating a response includes generating an alert based on the determined value representative of cardiac recovery, the activity type, activity intensity, the received input, or a combination thereof.

28. The method according to claim 27, wherein the alert is sent to a processor on a device associated with the subject.

29. The method according to claim 27, wherein the alert is sent to a processor on a device associated with a medical practitioner.

30. The method according to claim 27, wherein the alert is sent to emergency services personnel.

31. The method according to claim 27, wherein the alert is sent to a predefined person or group of people.

32. The method according to claim 27, wherein generating an alert includes identifying a potential issue based on the first value, the activity type, activity intensity, the received input, or a combination thereof.

33. The method according to claim 32, wherein the alert includes the potential issue.

34. The method according to claim 27, wherein the alert includes a location of the subject.

35. The method according to claim 25, further comprising determining a location based on the received input and storing the location.

36. The method according to claim 35, wherein the stored locations are configured to be accessed by a clinician and/or researcher.

37. The method according to claim 25, wherein the mechanical blood pump is inserted into a chamber of a subject's heart.

38. The method according to claim 25, wherein the one or more sensors comprises an accelerometer, a gyroscope, a heart rate sensor, and a pressure sensor.

39. The method according to claim 25, wherein the one or more sensors comprises a sensor in or on the mechanical blood pump.

40. The method according to claim 25, wherein the one or more sensors further comprises a sensor in or on a patch operably coupled to the subject.

41. The method according to claim 25, wherein the one or more sensors further comprises a sensor in a wearable device operably coupled to the subject.

42. The method according to claim 25, wherein determining the activity type comprises determining if a subject is engaged in a specific activity, the specific activities comprising walking, sitting, standing, lying down, and changing from lying down to sitting up.

43. The method according to claim 25, wherein a machine learning algorithm is used to determine the activity type and the activity intensity.

44. The method according to claim 25, further comprising adjusting a level of support and monitoring a parameter of heart recovery in response to the step of adjusting.

45. The method according to claim 44, further comprising generating a second response based on the monitored parameter of heart recovery.

46. The method according to claim 45, wherein the step of generating a second response includes an adjustment of a flow rate of the blood pump based on the monitored parameter of heart recovery.

47. A system for monitoring and supporting mechanical blood pumps in use with a subject, comprising:
a mechanical blood pump;
one or more sensors configured to detect a movement and a physiological condition of the subject; and
one or more processors configured to:
receive input from the one or more sensors;
determine an activity type, an activity intensity, or both an activity type and intensity based on the received input;
generate a response based on the activity type, activity intensity, the received input, and/or a combination thereof.
